(19) United States
(12) Patent Application Publication
(10) Pub. No.: US 2026/0174644 A1
(43) Pub. Date: Jun. 25, 2026

(54) ANTIPERSPIRANT STICK COMPOSITION

(71) Applicant: Conopco, Inc., d/b/a UNILEVER, Hoboken, NJ (US)

(72) Inventors: James Michael BIANCHI, Niles, IL (US); Yingxia WANG, Ansonia, CT (US); Manuel Alejandro ROSALES, Milford, CT (US)

(73) Assignee: Conopco, Inc., d/b/a UNILEVER, Hoboken, NJ (US)

(21) Appl. No.: 19/422,893

(22) Filed: Dec. 17, 2025

(30) Foreign Application Priority Data
Dec. 23, 2024 (EP) 24223056.3

(51) Int. Cl. A61K 8/26; A61K 8/28; A61K 8/31; A61K 8/37; A61K 8/86; A61K 8/92; A61Q 15/00

(52) U.S. Cl. CPC A61K 8/26; A61K 8/28; A61K 8/31; A61K 8/37; A61K 8/86; A61K 8/92; A61Q 15/00; A61K 2800/34

(57) ABSTRACT

An anhydrous antiperspirant stick composition comprising from 5 wt % to 50 wt % antiperspirant active from 10 wt % to 35 wt % of a hydrocarbon composition comprising C15 to C19 alkane. The method of manufacture of an anhydrous antiperspirant stick composition and non-therapeutic method of achieving a reduction in perspiration from the human body comprising the topical application of an anhydrous AP stick composition.

### FIELD OF THE INVENTION

The present invention relates to antiperspirant (AP) stick compositions having high efficacy and reduced environmental impact, in particular compositions which are free from volatile cyclic silicone oils.

### BACKGROUND OF THE INVENTION

There are numerous publications describing stick compositions with carrier oils that are used as alternatives to or in addition to volatile cyclic silicone oils.

WO 2024132396 A1 discloses the use of an anhydrous antiperspirant (AP) stick composition comprising an AP active which is an activated aluminium chlorohydrate or an activated aluminium sesquichlorohydrate, and a carrier oil which is free from volatile cyclic silicone oil and which comprises a C12-C16 branched chain alkane and an ether oil, wherein the carrier oil has a refractive index with a weighted mean within 0.13 of that of the AP active.

### SUMMARY OF THE INVENTION

It is an object of the present invention to provide an AP stick composition which is free from volatile cyclic silicone oils and which still delivers good sensory benefits. It is a further object of the present invention to provide an AP stick composition which delivers excellent antiperspirancy without undue staining of clothing worn in close proximity to the site of application of the composition.

It is a further object of the present invention to provide an AP stick composition which delivers suitable hardness.

In a first aspect of the invention there is provided an anhydrous antiperspirant (AP) stick composition comprising an AP active which is an activated aluminium chlorohydrate or an activated aluminium sesquichlorohydrate, and a carrier oil which is free from volatile cyclic silicone oil and which comprises C15-C19 alkanes and a mineral oil.

In a second aspect of the invention there is provided a method of manufacture of an anhydrous AP stick composition according to the first aspect of the invention.

In a third aspect of the invention there is provided a non-therapeutic method of achieving a reduction in perspiration from the human body comprising the topical application of an anhydrous AP stick composition according to the first aspect of the invention.

In an aspect of the invention closely related to the third aspect of the invention described immediately, there is provided the non-therapeutic use of an anhydrous AP stick composition according to the first aspect of the invention to reduce perspiration from the surface of the human body following topical application thereof.

### DETAILED DESCRIPTION OF THE INVENTION

Herein, the term “anhydrous” should be understood to mean “essentially anhydrous”, comprising less than 1 percent by weight of water, excluding any water that is chemically bound to solids within the composition, such as the AP active.

Herein, topical application refers to application to the skin of the human body, in particular to the underarm regions of the human body.

Herein, antiperspirant actives are materials that reduce perspiration on the skin of the human body, typically following topical application.

Herein, methods and uses should be understood to be cosmetic methods/uses, i.e. non-therapeutic methods/uses. Likewise, compositions of the invention are cosmetic compositions. Herein, all percentages, parts, and ratios are by weight, unless otherwise indicated. Herein, references to an amount of a material or materials refer to the total amount of material(s) of the type indicated.

Herein, references to an amount of a material are with reference to the total composition of which it is a part, unless otherwise indicated.

Herein, “application” and “applied” relate to application to the skin of the human body, in particular the underarm regions, unless the context dictates otherwise.

Herein, features expressed as “preferred” with regard to a particular aspect of the invention should be understood to be preferred with regard to each aspect of the invention. Herein, preferred, particularly preferred and especially preferred features of the invention are particularly preferred when used in combination with other preferred, particularly preferred, and especially preferred features of the invention.

Herein, “ambient conditions” refer to 20 degrees centigrade and 1 atmosphere pressure, unless otherwise indicated.

Herein, the word “comprising” is intended to mean “including” but not necessarily “consisting of” or “composed of.” In other words, the listed steps or options need not be exhaustive.

Herein, except in the examples or where otherwise explicitly indicated, all numbers in this description and claims indicating amounts of material, physical properties of materials and/or use are to be understood as modified by the word “about”.

Herein, numerical ranges expressed in the format “from x to y” are understood to include x and y. When for a specific feature multiple preferred ranges are described in the format “from x to y”, it is understood that all ranges combining the different endpoints are also contemplated.

Herein, refractive index (RI) is measured using methods in the art under ambient conditions.

The AP active used in the present invention is an activated aluminium chlorohydrate (AACH) and/or an activated aluminium sesquichlorohydrate (AASCH). The AP active is typically activated by heating in aqueous solution and/or treatment with a water-soluble calcium salt, and preferably with an amino salt, in addition to the water-soluble calcium salt.

In an alternative embodiment, the AP active used in the present invention may be a combination of aluminum halohydrates (especially chlorohydrates and zirconium chlorohydrates together with amino acids such as glycine which are disclosed in U.S. Pat. No. 3,792,068. Certain of those Al/Zr complexes are commonly called ZAG actives. ZAG actives generally contain aluminum, zirconium and chlorine with an Al/Zr ratio in the range of from 2 to 10, especially 2 to 6, and Al/Cl ratio of from 2.1 to 0.9 and a variable amount of glycine. Actives of this preferred type are available from manufacturers that include SummitReheis and Guilini. In one preferred embodiment, the antiperspirant salt in as activated aluminum zirconium tetrachlorohydrate complex with glycine, herein referred to as AAZG.

Herein, an “activated” aluminium chlorohydrate (ACH) or aluminium sesquichlorohydrate (ASCH) is one having an enhanced Band Ill content compared with the active before activation (vide infra). Heat activation of ACH is described in U.S. Pat. No. 4,359,456 (Gosling et al, Unilever, 1982).

Further improvements to this breakthrough technology were published subsequently. Activation by treatment with a water-soluble calcium salt and an amino salt or a hydroxy acid is described in WO 00/10512 (Gillette, 2000).

Heat activation of ASCH is described in U.S. Pat. No. 9,572,758 B2 (Gubrandsen, 2017).

A preferred AP active for use in the present invention is an ASCH activated by a water-soluble calcium salt and an amino acid. Such actives are described in EP 2,999,452 B1 (Unilever, 2017), EP 3,212,296 B1 (Unilever, 2019) and in other publications.

ASCH is an AP salt having the chemical formula Al2OH4·4Cl1.6 to Al2OH4.9Cl1.1. Most commercial ASCH samples are of chemical formula Al2OH4.7Cl1.3 to Al2OH4.9Cl1.1 and it is preferred to have ASCH salts of this formula.

The AP active is preferably formulated with a water-soluble calcium salt and an amino acid to achieve activation.

When employed, water-soluble calcium salt is preferably used at such a level that the molar ratio of calcium to aluminium is at least 1:40, more preferably at least 1:30 and most preferably at least 1:20. It is not advantageous to have the calcium concentration in excess of the aluminium concentration; indeed, it is preferred that the calcium concentration is no more than half that of the aluminium concentration and more preferred that it is no more than a fifth of said concentration. For the preferred molar ratios of calcium to aluminium of at least 1:40 and at least 1:20, it is independently preferred that this ratio is no greater than 1:2 and more preferred that it is no greater than 1:5.

In particularly preferred embodiments using calcium-activation, the molar ratio of calcium to aluminium is at least 1:15 and preferably no greater than 1:5 and in especially preferred embodiments it is at least 1:10 and preferably no greater than 1:5. A preferred water soluble calcium salt for use in calcium-activation is calcium chloride.

Herein, references to molar amounts and ratios of “aluminium” are calculated on the basis of mono-nuclear aluminium, but include aluminium present in poly-nuclear species; indeed, most of the aluminium in the salts of relevance is present in poly-nuclear species.

When amino acid is employed, it is preferably used at such a level that the molar ratio of amino acid to aluminium is at least 1:20, more preferably at least 1:10 and most preferably at least 1:5. It is not advantageous to have the amino acid concentration in excess of the aluminium concentration; hence, the molar amino acid to aluminium is preferably from 1:20 to 1:1, more preferably from 1:10 to 1:1 and most preferably from 1:5 to 1:1.

In particularly preferred embodiments, a molar ratio of amino acid to aluminium of at least 1:4 and preferably no greater than 1:1 is employed. In especially preferred embodiments it is at least 1:3 and preferably no greater than 1:1.

In preferred embodiments, a molar ratio of calcium to aluminium is at least 1:40 and the molar ratio of amino acid to aluminium is at least 1:20 is employed. In further preferred embodiments the molar ratio of calcium to aluminium is at least 1:20 and the molar ratio of amino acid to aluminium is at least 1:10. In particularly preferred embodiments the molar ratio of calcium to aluminium is from 1:20 to 1:5 and the molar ratio of amino acid to aluminium is from 1:10 to 1:1.

The above indicated preferences for calcium to aluminium molar ratio and/or amino acid to aluminium molar ratio lead to compositions of higher Band III content (vide infra) and, in general, higher antiperspirancy performance. It will be noted that higher Band III content is generally indicative of higher antiperspirancy performance.

When employed, preferred amino acids for use in the present invention are glycine, alanine, valine and proline. A particularly preferred amino acid for use in the present invention is glycine. The presence of the water-soluble calcium salt and amino acid lead to activation of the antiperspirant salt, i.e. an increase in its Band III content (vide supra).

In preferred embodiments, an aqueous solution of the AP salt is heated with the water-soluble calcium salt and glycine to achieve activation. The content of “Band III material” in an antiperspirant salt may be determined by SEC (Size Exclusion Chromatography) analysis. The SEC technique is well known in the art and is described in further detail in U.S. Pat. No. 4,359,456 (Gosling). The SEC band commonly referred to as Band III is designated as “Peak 4” in EP 1,104,282 B1 by Gillette.

Herein, “Band Ill content” refers to the integrated area in the Band III region of the SEC chromatograph relative to the total integrated area in all of the regions corresponding to aluminium species; that is to say, Bands I, II, III, and IV.

In preferred embodiments of the invention, compositions according to the invention have an AP active having a Band III content of at least 30 percent and more preferably at least 50 percent. The AP active used in the present invention is preferably a “milled” active, meaning that it has been ground down, typically to a relatively fine powder, and often following spray drying of an aqueous solution of the active. The milling produces an active that comprises non-hollow particles and this is a preferred feature of the invention, as it can aid in reducing white marks [see EP 2,999,452 B1 (Unilever, 2017)]. It is preferred that the AP active has an RI of from 1.52 to 1.57, more preferably 1.53 to 1.55 and most preferably about 1.54.

The milled AP active, when employed, preferably has a mean particle size (D50) of at least 6 microns, preferably from 6 to 25 microns, and more preferably from 7 to 15 microns.

Herein, mean (D50) particle sizes may be measured using (laser) light scattering techniques, for example using a Mastersizer instrument, obtainable from Malvern Instruments. Such instruments are set to produce a volume plot and a lens is selected in accordance with the maker's instructions to accommodate the expected particle size distribution, (or various lenses can be tested until the best lens is identified). Measurements are made by methods known in the art.

Herein, references to amounts of the AP active, including ratios relative thereto, refer to ASCH present as an anhydrous solid, i.e. excluding any water of hydration associated with the active, and also excluding the water-soluble calcium salt and the amino acid present in the composition and/or associated with the AP active.

The AP active is present in the composition at less than 50%, preferably less than 30%, more preferably less than 25%, and, in compositions giving especially low marks, less than 20%.

In order to achieve good to excellent AP efficacy, it is also important not to have too little AP active present. Hence, the level of AP active is preferably from 5 to 50%; from 5 to 30 percent, more preferably from 10 to 30 percent and further preferably from 12 to 25 percent. For compositions giving especially low marks, the most preferred level is from 12 to 20 percent.

Compositions according to the present invention have a certain hardness in order for them to be used as sticks or soft solids.

The “hardness” of stick and soft solid compositions refers to the depth, in millimetres, that a cone penetrates into a test specimen under fixed conditions, as determined in accordance with the procedures of ASTM Method D217-48, incorporated herein by reference, using a Petrotest PNR 10 Penetrometer (or equivalent), equipped with an ASTMD2884 plunger (Petrotest Cat. #18-0081 or equivalent, weight=47.5 g) and a 2.5 g aluminium cone, 20° angle with a base diameter of 10 mm, wherein hardness values are reported as an average of 6 replicate measurements.

Compositions according to the present invention typically have a hardness of less than 30 mm, preferably less than 25 mm and more preferably less than 20 mm.

Soft solid compositions according to the present invention typically have a hardness value of from 5 to 25 mm.

Stick compositions according to the present invention typically have a hardness value of less 20 mm and preferably less than 15 mm. Particularly preferred stick compositions have a hardness of from 5.5 to 12.5 mm.

Herein, the carrier oil is a liquid material that is non-miscible with water. It is free from volatile cyclic silicone oil and comprises a C15-C19 alkane and mineral oil. Herein, volatile cyclic silicone oils and volatile silicones should be understood to be silicone oils having a vapour pressure of greater than 1 Pa at 25 degrees centigrade.

The absence of volatile cyclic silicone oil is essential for many reasons, including environmental benefits. It is preferred that compositions according to the invention are free from all volatile silicone oils and, more preferably, free from all silicone oils. By avoiding silicone oils, the biodegradability of the composition may be increased.

The C15-C19 alkane hydrocarbon is present to act as a carrier fluid/oil, either by itself or in combination with other carrier fluid/oil materials. Hence, compositions according to the invention may alternatively be described as comprising a carrier oil comprising a C15-C19 alkane hydrocarbon.

The C15-C19 alkane hydrocarbon may be present as a single material or it may comprise more than one material of that description.

The carrier fluid as whole, comprising the C15-C19 alkane hydrocarbon, is a liquid material that is non-miscible with water. It is preferably free from silicone oil.

The absence of silicone oil is preferable for many reasons, including environmental benefits. By avoiding silicone oils, the biodegradability of the composition may be increased.

In preferred embodiments, the C15-C19 alkane hydrocarbon is saturated. In particularly preferred embodiments, it comprises saturated C15-C19 linear, branched and cyclic hydrocarbons. In especially preferred embodiments, it consists of saturated C15-C19 linear, branched and cyclic hydrocarbons, the CAS number is 64742-46-7. Such materials are available from SEPPIC under the tradename Emosmart L19.

In preferred embodiments, the C15-C19 alkane hydrocarbon comprises from 10 to 35% by weight of the oils present in the carrier oil, preferably from 12 to 30% by weight of the oils present in the carrier oil, preferably from 15 to 25% by weight of the oils present in the carrier oil.

In preferred embodiments, the C15-C19 alkane hydrocarbon comprises from 10 to 35% by weight of the AP stick composition, preferably from 15 to 25% by weight of the AP stick composition.

An additional component of the carrier oil is a mineral oil, preferably a light mineral oil. An exemplary light mineral oil has a CAS number 8042-47-5.

In preferred embodiments, the mineral oil comprises from 10 to 35% by weight of the oils present in the carrier oil, preferably from 12 to 30% by weight of the oils present in the carrier oil, preferably from 15 to 25% by weight of the oils present in the carrier oil.

In a preferred embodiment the C15-C19 alkane hydrocarbon and mineral oil are employed, preferably a light mineral oil.

In a preferred embodiment the ratio of C15-C19 alkane hydrocarbon and mineral oil is from 3:1 to 1:3, preferably from 2:1 to 1:2.

Further suitable carrier oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C12-15 alkyl benzoate, for example Finsolv TN™ or Finsolv Sun™.

In some preferred embodiments, an ester oil is also employed. A particularly preferred type of ester oil is an alkyl ether of an aryl group, such as C12-C15 alkyl benzoate.

In preferred embodiments, the ester oil comprises from 5 to 35% by weight of the oils present in the carrier oil, preferably from 8 to 25% by weight of the oils present in the carrier oil, preferably from 10 to 20% by weight of the oils present in the carrier oil.

Another type of ester oil used in certain preferred embodiments are triglycerides of unsaturated fatty acids. These oils are typically natural oils and are biodegradable. Herein, a “triglyceride of an unsaturated fatty acid” should be understood to mean a triglyceride wherein at least 50 percent of the fatty acids contain at least one double-bond. Such triglycerides are typically mixtures of glycerol esters with different fatty acids. A preferred oil of this type is Helianthus Annuus seed oil, otherwise known as sunflower seed oil.

A preferred additional component of the carrier fluid is an ether oil.

Suitable carrier oils can be selected from alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

In preferred embodiments, the ether oil comprises from 10 to 35% by weight of the oils present in the carrier oil, preferably from 12 to 30% by weight of the oils present in the carrier oil, preferably from 15 to 25% by weight of the oils present in the carrier oil.

In some preferred embodiments, an ester oil is also employed, especially in compositions also comprising an ether oil.

Another type of ester oil used in certain preferred embodiments are triglycerides of unsaturated fatty acids. These oils are typically natural oils and are biodegradable. Herein, a “triglyceride of an unsaturated fatty acid” should be understood to mean a triglyceride wherein at least 50% of the fatty acids contain at least one double-bond. Such triglycerides are typically mixtures of glycerol esters with different fatty acids. A preferred oil of this type is Helianthus Annuus seed oil, otherwise known as sunflower seed oil.

A further essential component of compositions of the invention is a thickening agent, sometimes alternatively referred to as a gelling agent or gellant. Such agents increase the viscosity of or solidify the carrier oil in which the particulate antiperspirant active is typically suspended.

The thickening agent may be selected from any of those known in the art. Often, the thickening agent includes a wax. Waxes typically are considered to melt at above 40° C. and particularly between 55 and 95° C. Waxes can include ester waxes, including C12 to C24 linear fatty alcohols, waxes obtained from animals or plants, often after hydrogenation, silicone elastomers and silicone waxes. The thickening agent can comprise a mixture of particulate thickening agents, a mixture of waxes or a mixture of both types of material.

The thickening agents used in compositions according to the invention, and especially stick compositions according to the invention, are preferably selected from fibre-forming non-polymeric gelling agents and waxes, optionally supplemented by particulate silica and/or an oil-soluble polymeric thickening agent.

Waxes employed herein as thickening agents are often selected from hydrocarbons, linear fatty alcohols, silicone polymers, esters of fatty acids or mixtures containing such compounds along with a minority (less than 50% w/w and often less than 20% w/w) of other compounds.

Naturally occurring waxes are often mixtures of compounds which include a substantial proportion of fatty esters.

Examples of hydrocarbon waxes include paraffin wax, ozakerite, microcrystalline wax and polyethylene wax, the last named desirably having an average molecular weight of from 300 to 600 and advantageously from 350 to 525. A preferred hydrocarbon wax is hydrogenated castor oil, also known as hydrogenated castor wax.

Linear fatty alcohols commonly contain from 14 to 40 carbon atoms and often from 16 to 24. Preferred thickening agents of this class are stearyl alcohol and behenyl alcohol, with stearyl alcohol being especially preferred.

In preferred embodiments, the stearyl alcohol comprises from 10 to 35% by weight of the oils present in the carrier oil, preferably from 12 to 30% by weight of the oils present in the carrier oil, preferably from 15 to 25% by weight of the oils present in the carrier oil.

Examples of ester waxes include esters of C16-C22 fatty acids with glycerol or ethylene glycol, which can be isolated from natural products or more conveniently synthesized from the respective aliphatic alcohol and carboxylic acid.

Examples of natural waxes include beeswax, wool wax and spermaceti wax of animal origin, and caster wax, jojoba wax, carnauba wax and candelilla wax which are of vegetable origin. Montan wax, which is an example of mineral wax, includes non-glyceride esters of carboxylic acids, hydrocarbons and other constituents.

Fibre-forming thickening agents are dissolved in the carrier oil at elevated temperature and on cooling precipitate out to form a network of very thin strands that structure, i.e. thicken, the carrier oil. One particularly effective category comprises N-acyl aminoacid amides and in particular linear and branched N-acyl glutamic acid dialkylamides, such as in particular N-lauroyl glutamic acid di n-butylamide and N-ethylhexanoyl glutamic acid di n-butylamide and especially mixtures thereof. Such amido gellants can be employed in anhydrous compositions according to the present invention, if desired, with 12-hydroxystearic acid.

Other amido SMGAs include 12-hydroxystearic acid amides, and amide derivatives of di and tribasic carboxylic acids as set forth in WO 98/27954, including notably alkyl N,N′dialkyl succinamides.

Further suitable amido-containing thickening agents are described in U.S. Pat. Nos. 6,410,003, 7,332,153, 6,410,001, 6,321,841, and 6,248,312.

The thickening agent is typically employed in the composition at a concentration of from 1.5 to 30%. When a fibre-forming thickening agent is employed, its concentration is typically in the range of from 1.5 to 15%. When a wax is employed, its concentration is usually selected in the range of from 0.1 to 10%, and particularly from 0.5 to 6% w/w.

Some highly desirable compositions comprise in combination a first thickening agent with a second thickening agent.

One category of oil-soluble polymer thickening agent which has been found suitable is a polysaccharide esterified with a mono-carboxylic acid containing at least 12 carbon atoms, and preferably a dextrin fatty acid ester such as dextrin palmitate or dextrin stearate. Commercial products are available under the trade mark Rheopearl.

A second category of polymer thickening agent comprises polyamides for example those discussed in U.S. Pat. No. 5,500,209 or U.S. Pat. No. 6,353,076.

A third category of thickening agent comprises block copolymers of styrene with ethylene propylene and/or butylene available under the trade name KRATON, and particularly styrene ethylene/butylene styrene linear block copolymers. A related category of thickening polymer comprises polymers of alpha methylstyrene and styrene, such as those under the trade name KRISTALEX, eg KRISTALEX F85 having a mean molecular weight of approximately 1200. Yet another thickening polymer comprises alkyl substituted galactomannan available under the trade name N-HANCE AG.

A still further class of thickening polymers co-polymers of vinyl pyrrolidone with polyethylene containing at least 25 methylene units, such as triacontanyl polyvinylpyrrolidone, under the trade name Antaron WP-660.

Such thickening polymer is often employed in a weight ratio to the oil blend that is selected in the range of from 1:30 to 1:5, taking into account the hardness of the soft solid that is desired, the inherent ability of the chosen polymer to increase viscosity and the presence or otherwise of an additional thickening agent.

A thickening agent especially well suited to forming or contributing to the formation of a soft solid composition comprises particulate silica and especially fumed silica. It is desirable to include at least 2% and especially at least 2.5% by weight of the silica in the composition, such as in the range of up to 10% by weight.

Other components that may be included in compositions according to the invention including those described in the following paragraphs.

Wash-off agents may be included, often in an amount of up to 10%, to assist in the removal of the formulation from skin or clothing. Such wash-off agents are typically non-ionic surfactants such as esters or ethers containing a C₈ to C₂₂ alkyl moiety and a hydrophilic moiety comprising a polyoxyalkylene group (POE or POP).

Skin feel improvers (e.g. talc or finely divided high molecular weight polyethylene), may be included, typically in an amount from 1 up to 10%.

Skin moisturisers, such as glycerol or polyethylene glycol (e.g. mol. wt. 200 to 600) may be included, typically in an amount of up to 5%.

Skin benefit agents, such as allantoin or lipids, may be included, typically in an amount of up to 5%.

A highly preferred optional component is a preservative, such as ethyl or methyl parabens or BHT (butyl hydroxy toluene), typically in an amount of from 0.01 to 0.1%.

Herein, the terms “oil” and signifies a water-insoluble organic material that is liquid at 20° C. Any material having a solubility of less than 0.1 g/100 g at 20° C. is considered to be insoluble.

The carrier oil as a whole and the components thereof preferably have good sensory properties. This can be important to consumers during and/or after topical application of the composition.

A preferred additional component for use in compositions of the present invention is a fragrance or fragrance oil, sometimes alternatively called a perfume (oil). The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the total composition is preferably up to 3 percent, advantageously is at least 0.5 percent and particularly from 0.8 percent to 2 percent. The amount of fragrance oil in the composition, excluding any propellant that may be present therein, is preferably up to 20 percent, advantageously is at least 4 percent and particularly from The method of manufacture involves multiple steps; in particular, steps involved in the manufacture of the AP active and steps involved in its formulation.

In preferred embodiments, an AP active, which is preferably ASCH, is first activated by heating it in aqueous solution with a water-soluble calcium salt and an amino acid.

Calcium chloride and glycine may be used as the water-soluble calcium salt and the amino acid in this step. The aqueous mixture is typically heated to an extent to achieve a Band III content of at least 55 percent. The aqueous mixture may be heated to a temperature of greater 70 degrees centigrade for greater than 3 hours.

Following the activation step described immediately above, the aqueous solution is typically spray dried.

Following the spray drying, the AP active is milled, preferably to give a mean particle size (D50) of from 6 to 25 microns, and more preferably from 7 to 15 microns. The milled AP active is formulated into the stick composition by first mixing it with the carrier oils comprising C15-C19 alkane hydrocarbon. This is preferably done using a high shear mixer. Fragrance may than be added.

A preferred method of manufacture combines some or all of the processes described in the above paragraphs. An exemplary method of preparing antiperspirant stick products is described in: ‘Chemistry and Technology of the Cosmetics and Toiletries Industry’ 2^{nd} Ed., Blackie Academic & Professional (1996); section 10.7.2.

### EXAMPLES

The stick compositions described in Table 1 were prepared by methods known in the art. An exemplary method of preparing antiperspirant stick products is described in: ‘Chemistry and Technology of the Cosmetics and Toiletries Industry’ 2^{nd} Ed., Blackie Academic & Professional (1996); section 10.7.2.

A study of white marks produced by a formulation according to invention vs. white marks produced by a formulation comprising a cyclic volatile silicone (cyclopentasiloxane) instead of C15-C19 alkane and mineral oil was performed using the compositions detailed in Table 1.

The compositions were wiped onto black cloths from the same distance and using the same amount. Example 1 produced greatly reduced white marks when compared with Comparative Example A.

TABLE 1

| | Ex A | |
|---|---|---|
| INCI Name | comparative | Ex 1 |
| Cyclopentasiloxane | 20 | 0 |
| C15-19 Alkane | 0 | 13.6 |
| Stearyl Alcohol | 18 | 18 |
| Hydrogenated Castor Oil | 4 | 4 |
| Synthetic Wax/Polyethylene | 1 | 1 |
| Isopropyl Palmitate | 12 | 0 |
| C12-15 Alkyl Benzoate | 15 | 10 |
| PPG-14 Butyl Ether | 10 | 18 |
| Helianthus Annuus Seed Oil | 0.5 | 0 |
| Dimethicone | 1 | 0 |
| Light Mineral Oil | 0 | 10.6 |
| Butylated Hydroxytoluene | 0.05 | 0 |
| PEG-8 | 2 | 0 |
| Steareth-100 | 0.5 | 0.3 |
| Silica | 1 | 0 |
| Aluminium Sesquichlorohydrate, | 15 | 24.5 |
| Calcium Chloride, Glycine, Aqua | | |
| | 100 | 100 |
| Hardness | 5.5 | 6.5 |

Ex A and Ex 1 have parity scores for white marks. Ex 1 shows that a carrier oil comprising C15-C19 alkane and light mineral oil results in a stick composition with 60 wt % greater antiperspirant active ingredient, significantly increasing the antiperspirant activity of the stick composition without increasing the amount of antiperspirant active present in the composition.

Furthermore, comparable hardness is achieved.

1. An anhydrous antiperspirant stick composition comprising:
a from 5 wt % to 50 wt % antiperspirant active
b from 10 wt % to 35 wt % of a hydrocarbon composition comprising C15 to C19 alkane and
c a mineral oil.

2. A stick composition according to claim 1, wherein the wherein the weight ratio of C15 to C19 alkane to mineral oil is from 2:1 to 1:2.

3. A stick composition according to claims 1 to 2, wherein the composition is free from silicone oil.

4. A stick composition according to claims 1 to 3, wherein the antiperspirant active composition comprises an aluminum halohydrate salt or aluminum zirconium tetrachlorohydrate salt.

5. A stick composition according to claims 1 to 4, wherein the antiperspirant active composition comprises activated aluminum sesquichlorohydrate.

6. A stick composition according to claims 1 to 5, comprising an ester.

7. A stick composition according to claim 6, wherein the ester is C12-15 Alkyl Benzoate.

8. A stick composition according to claims 1 to 7 comprising an ether.

9. A stick composition according to claim 8, wherein the ether is PPG-14 Butyl Ether.

10. A stick composition according to claims 1 to 9 comprising a thickening agent.

11. A stick composition according to claims 1 to 9 comprising a wax.

12. A method of manufacture of a stick composition according to one any of claims 1 to 11.

13. A non-therapeutic method of achieving a reduction in perspiration from the human body comprising the topical application of an anhydrous AP stick composition according to claims 1 to 11.
